(19) United States
(12) Patent Application Publication
(10) Pub. No.: US 2014/0277105 A1
(43) Pub. Date: Sep. 18, 2014

(54) MATERIAL MANIPULATION METHOD AND APPARATUS

(71) Applicant: Vanderbilt University, Nashville, TN (US)

(72) Inventors: Ray Lathrop, Nashville, TN (US); Richard Hendrick, Nashville, TN (US); John S. Schneider, Nashville, TN (US); Robert J. Webster, Nashville, TN (US)

(73) Assignee: Vanderbilt University, Nashville, TN (US)

(21) Appl. No.: 14/177,735

(22) Filed: Feb. 11, 2014

Related U.S. Application Data
(60) Provisional application No. 61/787,001, filed on Mar. 15, 2013.

(51) Int. Cl. A61B 17/28

(52) U.S. Cl. CPC A61B 17/28
USPC 606/205

(57) ABSTRACT

A method and apparatus for use in manipulating material during a medical procedure includes positioning the material in engagement with a major side surface on a distal end portion of a shaft. A gripper is operated to grip the material between a wire and a major side surface of the shaft by moving the wire along a longitudinal central axis of the shaft. Material is moved to a desired position relative to a patient's body while the material is gripped between the wire and the major side surface of the distal end portion of the shaft. The wire is then retracted along the longitudinal central axis of the shaft to release the material for movement relative to the end distal end portion of the shaft.

### RELATED APPLICATION

This application claims the benefit of U.S. Provisional Patent Application Ser. No. 61/787,001, filed Mar. 15, 2013, the subject matter of which is incorporated hereby incorporated by reference in its entirety for all purposes.

### TECHNICAL FIELD

The present disclosure relates to a new and improved method and apparatus for use in manipulating material during a medical procedure.

### BACKGROUND

Material has previously been manipulated during a medical procedure using a grasper having symmetric jaws to initially place the material, such as graft material, into a hole in a receiving area in bone tissue. Positioning of the small, light graft material is very sensitive and easily disturbed by small movements of the surgical tools. Typical tools used to place the graft material are endoscopic forceps and spatula like probes for fine positioning. Placing of the graft material typically requires the forceps jaws to lie alongside the skull base where the graft material is being placed. Releasing/opening the jaws requires clearance between the graft material and receiving tissue. Thus, opening the forceps will move the material and additional time will be required to finish positioning the material.

### SUMMARY

The present disclosure relates to new and improved method and apparatus for manipulating material during a medical procedure.

Material to be positioned during the medical procedure is placed in engagement with a major side surface on a distal end portion of a shaft which extends from a handle. A gripper is movable along a longitudinal central axis of the shaft to grip the material between the gripper and the major side surface of the distal end portion of the shaft. A shoulder may be provided between a distal end portion of the shaft and a proximally extending portion of the shaft to retard movement of the material in a proximal direction. A distal end portion of the shaft is shaped so that it can be used as a spatula for fine positioning of the material.

It is contemplated that the method and apparatus of the present disclosure will be utilized with many different types of materials. The method and apparatus may be used to position graft material relative to a patient's body. Alternatively, the method and apparatus will be used to place a drug/medicine in some hard-to-access location. Of course, the method and apparatus may be used to position other known materials.

### BRIEF DESCRIPTION OF THE DRAWINGS

The foregoing and other features of the present disclosure will become more apparent upon a consideration of the following description taken in connection with the accompanying drawings wherein:

FIG. 1 is a schematic pictorial illustration of a tool which is constructed in accordance with the present disclosure and operated to manipulate material during a medical procedure;

FIG. 2 is an enlarged fragmentary schematic illustration depicting the manner in which material is positioned relative to a patient's body using the tool of FIG. 1;

FIG. 3 is an enlarged schematic pictorial illustration of a distal end portion of the tool of FIG. 1 and illustrating the relationship between a shaft and a material gripper;

FIG. 4 is an enlarged schematic pictorial illustration depicting the manner in which the tool of FIGS. 1 and 3 is utilized to grip material during a medical procedure;

FIG. 5 is an enlarged schematic fragmentary illustration of a second embodiment of the distal end portion of the tool of FIG. 1;

FIG. 6 is an enlarged schematic pictorial illustration of a third embodiment of the distal end portion of the tool of FIG. 1;

FIG. 7 is an enlarged schematic pictorial illustration of a fourth embodiment of the distal end portion of the tool of FIG. 1;

FIG. 8 is an enlarged exploded schematic pictorial illustration of the tool of FIG. 1; and

FIG. 9 is an enlarged schematic illustration further depicting the construction and mode of operation of the tool of FIG. 1.

### DETAILED DESCRIPTION

A tool 10 (FIG. 1) is constructed in accordance with one aspect of the present disclosure and is utilized to manipulate material during a medical procedure. The tool 10 includes a handle assembly 12 and a shaft 14 which extends from the handle assembly 12. A proximal end portion 16 of the shaft 14 is fixedly connected with the handle assembly 12.

The shaft 14 has a distal end portion 18 (FIGS. 1-3), which is engagable with material 22 to be positioned during a medical procedure. In the specific embodiment illustrated in FIG. 2, the material 22 is bone graft. The bone graft 22 or other material is positioned relative to a portion 24 of a patient's body (FIG. 2) by the tool 10.

Although the material 22 illustrated in FIG. 2 is bone graft, it is contemplated that the tool 10 may be utilized to position many different types of material relative to a patient's body. For example, the tool 10 may be utilized to position a drug/medicine loaded patch relative to a hard-to-access location in a patient's body. Alternatively, the tool 10 may be utilized to place graft material, other than bone graft, relative to a patient's body. It is contemplated that the tool 10 may be utilized to position material relative to either a human or animal patient's body.

In accordance with one of the features of the present disclosure, the material 22 is gripped against a major side surface 26 (FIG. 3) on an expanded portion 28 (FIG. 3) of the distal end portion 18 of the shaft 14. The expanded end portion 28 of the shaft 14 may be formed by at least partially flattening the distal end portion 18 of the shaft 14 in the manner illustrated in FIG. 3.

A gripper 32 extends across the expanded portion 28 to grip the material 22 between the major side surface 26 of the expanded portion and the gripper (FIGS. 3-4). In the embodiment of the present disclosure illustrated in FIGS. 1-4, the gripper 32 is a resiliently deflectable wire 36. The wire 36 extends along the tubular shaft 14 from the distal end portion 18 of the shaft 14 and through the proximal end portion 16 (FIG. 1) of the shaft 14 to a rocker member or actuator 38 (FIGS. 7-8) which is pivotally connected to the handle assembly 12 (FIGS. 7-8).

When the actuator 38 is pivoted in a counterclockwise direction, as viewed in FIGS. 7 and 8, the wire 36 is moved along the longitudinal central axis of the shaft 14 to a retracted position. When the wire 36 is in the retracted position, a distal end portion 44 of the wire is withdrawn into the shaft 14. At this time, the wire 36 is disposed to the right (as viewed in FIG. 3) of the major side surface 26 of the expanded portion 28 of the shaft 14. The distal end portion 44 of the wire 36 is almost completely enclosed by the tubular shaft 14.

When the actuator 38 is pivoted in a clockwise direction (as viewed in FIGS. 7-8) with the wire 36 in a retracted position, the wire is moved along the longitudinal axis of the shaft 14 from the retracted position to the extended position illustrated in FIGS. 3-4. In the illustrated embodiment of the present disclosure, the shaft 14 has a hollow cylindrical configuration and the wire 36 is positioned in the shaft 14 with a longitudinal central axis of the wire 36 coincident with the longitudinal central axis of the shaft. The cylindrical inner side surface of the shaft 14 engages a cylindrical outer side surface of the wire 36 to guide movement of the wire between the retracted and extended positions.

Although the wire 36 is positioned in the shaft 14 with the central axis of the wire adjacent to the central axis of the shaft, it is contemplated that the shaft may be constructed in such a manner that the longitudinal central axis of the wire 36 is offset to one side of the longitudinal central axis of the shaft. The shaft 14 has an end portion 50 which can be manually bent to any desired configuration. This enables the shaft 14 and the resiliently deflectable wire 36, which is enclosed by the shaft, to be bent to a desired configuration immediately prior to usage of tool 10. In the illustrated embodiment of the tool 10, only the end portion 50 is readily bent to a desire configuration. A body portion 54 of the shaft 14 has a greater rigidity than the end portion 50 and is not bent manually. However, it is contemplated that the entire shaft 14 may be formed so that it can be readily bent manually to a desired configuration immediately before it is used to perform a particular procedure.

The resiliently deflectable wire 36 is formed of a nickel titanium alloy (Nitinol). However, the wire 36 may be formed of other materials. For example, the wire 36 may be formed of stainless steel. Alternatively, the wire 36 may be formed of a suitable polymeric material.

In the embodiment illustrated in FIGS. 1-4, the distal end portion 44 of the wire 36 is axially tapered. This facilitates movement of the wire across an upper (as viewed in FIG. 4) side surface 58 of the material 22. A lower side surface of the material 22 is pressed against the major side surface 26 on the expanded portion 28 of the shaft 14 (FIG. 3) by the resilient wire 36. Of course, the distal end portion 44 of the wire 36 may have the same cylindrical configuration as the remainder of the wire and not be axially tapered.

When the material 22 is to be gripped by the tool 10, the actuator 38 is pivoted in a counterclockwise direction (as viewed in FIGS. 7-8) to move the wire 36 to its retracted position. This results in the distal end portion 44 of the wire being telescopically moved into the shaft 14 and the major side surface 26 of the expanded portion 28 exposed. The material 22 is then positioned with its lower side surface in abutting engagement with the major side surface 26 of the expanded portion 28.

While the material 22 is in engagement with the major side surface 26 of the expanded portion 28 of the shaft 14, the actuator is pivoted in a clockwise direction (as viewed in FIGS. 7 and 8) to move the wire 36 longitudinally from its retracted position to the extend position illustrated in FIG. 3. As this occurs, the distal end portion 44 of the wire 36 slides along the upper side surface 58 (FIG. 4) of the material 22. The material 22 is gripped between the wire 36 and the upwardly (as viewed in FIG. 3) facing major side surface 26 of the expanded portion of the shaft 14.

At this time, a proximal or trailing end portion 62 of the material 22 (FIG. 4) is disposed in engagement with a shoulder 64 formed between the expanded portion 28 of the shaft 14 and the cylindrical end portion 50 of the shaft. The shoulder 64 positions the material 22 on the major side surface 26 of the expanded portion 28 (FIG. 3) of the shaft 14.

Once the material 22 has been gripped between the expanded portion 28 of the shaft 14 and the wire 36, the tool 10 is manually moved by an operator who is engaging the handle assembly 12. The operator moves the tool 10 to position the material 22 in a desired location relative to a patient's body. As the tool 10 is moved, the wire 36 holds the material 22 against movement relative to the major side surface 26.

Once this has occurred, the actuator member 38 is pivoted in a counterclockwise direction (as viewed in FIGS. 7-8) to move the wire 36 toward its retracted position. As the wire 36 moves toward its retracted position, the material 22 is gradually released for movement to a desired position in a patient's body. The flat major side surface 26 on the expanded portion 28 is utilized to press the material against the patient's body at the desired location. Once the wire 36 has been moved to a retracted position within the shaft 14, the expanded portion 28 of the shaft 14 is used as a spatula to press the material firmly into the desired location in the patient's body. This may be done by applying pressure against the material with the flat major side surface 26 of the expanded portion 28. In addition, a flat lower major side surface 68 (FIG. 3) of the expanded portion 28 may be pressed against the material 22 with a wiping action.

In the embodiment of the present disclosure illustrated in FIGS. 1-4, the expanded portion 28 of the shaft 14 has flat upper and lower major side surfaces 26 and 68. However, it is contemplated that the expanded portion 28 may have arcuately curving major side surfaces. Thus, the upper major side surface 26 may have a concave configuration while the lower major side surface 68 has a convex configuration. Alternatively, the expanded portion 28 of the shaft 14 may have an upwardly facing major side surface 26 with a flat section in a central portion of the major side surface and upwardly (as viewed in FIG. 3) extending edge portions extending along and spaced from opposite sides of the wire 36. These edge portions would extend parallel to the longitudinal central axis of the wire 36 and would facilitate gripping of the material 22 between the wire and the expanded portion 28 of the shaft 14.

Although it is contemplated that the material 22 may be any one of many different materials, in the embodiment of the present disclosure illustrated in FIG. 2, the material 22 is bone graft material which is being positioned in a recess 74 formed in a base 76 of a patient's skull. The flat upwardly facing major side surface 26 (FIG. 3) on the expanded portion 28 of the shaft 14 is utilized to press against the bone graft material to position it in the recess 74 (FIG. 2). The shaft 14 may then be rotated and the flat lower side surface 68 on the expanded portion 28 of the shaft is used to further press the bone graft material into the desired position relative to the patient's skull. If desired, the gripper 32 may be retracted and only the major side surface 26 of the expanded portion 28 of the distal end portion 18 of the shaft 14 used as a tool to press the bone graft material in place.

In the embodiment of the present disclosure in FIGS. 1-4, the expanded portion 28 has a flat major side surface 26 and the gripper 32 extends for only a relatively short distance past the expanded portion 28 when the gripper is in the extended position. In the embodiment of the present disclosure illustrated in FIG. 5, the expanded portion has an arcuate major side surface and the gripper extends a substantial distance past the expanded portion. Since the embodiment of the present disclosure illustrated in FIG. 5 is generally similar to the embodiments of the present disclosure in FIGS. 1-4, similar numerals will be utilized to identify similar components, the suffix letter “a” being added to the numerals of FIG. 5 to avoid confusion.

A tool 10a (FIG. 5) is used to manipulate material during a medical procedure. The tool 10a includes a handle assembly (not shown in FIG. 5) corresponding to a handle assembly 12 of FIG. 1. A shaft 14a extends from the handle assembly. The shaft 14a has a distal end portion 18a. An expanded portion 28a is provided at the distal end portion 18a. A gripper 32a extends across an arcuate major side surface (not shown) formed on the expanded portion 28a. The gripper 32a is formed by a wire 36a which is connected with the handle assembly in the manner previously explained in conjunction with the embodiment of the present disclosure illustrated in FIGS. 1-4.

In the embodiment of the present disclosure illustrated in FIG. 5, the expanded portion 28a has a semi-circular configuration. This results in the major side surface on the expanded portion 28a having the configuration of a portion of a cylinder. The cylindrical wire 36a has a diameter which is less than the diameter of the semi-circular major side surface on the expanded portion 28a. Wire 36a extends past the end of the expanded portion 28a for a substantial distance.

In the embodiment of the present disclosure illustrated in FIGS. 1-4, a gripper 32 (FIG. 3) is formed by wire 36 having a tapered end portion. In the embodiment of the present disclosure illustrated in FIG. 6, a gripper is formed by a wire having an expanded end portion. Since the embodiment of the present disclosure illustrated in FIG. 6 is generally similar to the embodiments of the present disclosure illustrated in FIGS. 1-5, similar numerals will be utilized to identify similar components, the suffix letter “b” being associated with the numerals of FIG. 6 to avoid confusion.

The tool 10b having the same general construction and mode of operation as the tool 10 of FIG. 1, includes a shaft 14b which extends from a handle assembly corresponding to the handle assembly 12 of FIG. 1. The shaft 14b (FIG. 6) has a distal end portion 18b with an expanded end portion 28b. The expanded end portion 28b has an upwardly (as viewed in FIG. 6) facing major side surface 26b. The major side surface 26b is flat and is planar. The expanded end portion 28b is integrally formed with the shaft 14 and is formed by expanding the distal end portion 18b of the shaft.

A gripper 32b extends from the handle assembly to the distal end portion 18b of the shaft 14b. The gripper 32b (FIG. 6) is formed by a resiliently deflectable wire 36b. In accordance with one of the features of this embodiment of the disclosure, the wire 36b has an expanded end portion 82. The expanded end portion 82 of the resilient wire 36b may be flat so that it is generally parallel to the upwardly facing major side surface 26b of the expanded portion 28b of the shaft 14b.

However, the illustrated expanded end portion 82 of the gripper 32b has an arcuately curving configuration. The expanded end portion 82 of the wire 36b has an arcuately curving upper (as viewed in FIG. 6) major side surface 84 which faces away from the major side surface 26b of the expanded portion 28b of the shaft 14b. In addition, the expanded portion 82 of the wire 36b has a downwardly (as viewed in FIG. 6) facing and arcuately curving major side surface 88 which faces toward the relatively flat major side surface 26b formed on the expanded portion 28b of the shaft 14b. The axially outwardly and upwardly (as viewed in FIG. 6) curving lower major side surface 88 of the expanded portion 82 of the wire 36b cooperates with the relatively flat upwardly facing major side surface 26b of the expanded portion 28b of the shaft 14b to form a nip 92 where the arcuate lower major side surface 88 of the expanded end portion 82 of the wire 36b engages the flat major side surface 26b of the expanded end portion 28b of the shaft 14b.

The material 22 to be held by the tool 10b may be inserted into the nip 92 with a camming action which resiliently deflects the wire 36b slightly upwardly (as viewed in FIG. 6). This results in the material 22 being firmly gripped between the lower major side surface 88 on the expanded end portion 82 of the wire 36b and the upwardly facing major side surface 26b on the expanded end portion 28b of the shaft 14b.

If desired, the expanded end portion 28b of the shaft 14b may have an arcuate curving configuration for a portion of its length. Thus, the distally outer end portion of the expanded portion 28b of a shaft 14b may curve downwardly (as viewed in FIG. 6) away from the expanded end portion 82 of the wire 36b. This results in at least a portion of the upwardly facing major side surface 26b of the expanded end portion 28b of the shaft 14b having an arcuate configuration which would result in the nip 92 being formed between two arcuate surfaces, that is, the arcuately downwardly (as viewed in FIG. 6) curving major side surface 26b of the expanded portion 28b of the shaft 14b and the arcuately upwardly (as viewed in FIG. 6) curving lower major side surface 88b of the expanded portion 82 of the wire 36b. Of course, both the upwardly facing major side surface 26b of the expanded portion 28b of the shaft 14b and the upwardly facing major side surface 84 of the wire 36b may be flat and extend generally parallel to each other.

In the embodiment of the present disclosure illustrated in FIGS. 1-4, the wire 36 extends straight across the expanded portion 28 of the shaft 14. In the embodiment of the present disclosure illustrated in FIG. 7, the wire is formed with a bent end portion. Since the embodiment of the present disclosure illustrated in FIG. 7 is generally similar to the embodiment of the present disclosure illustrated in FIGS. 1-4, similar numerals will be utilized to designate similar components, the suffix letter “c” being added to the numerals of FIG. 7 to avoid confusion.

A tool 10c having the same general construction and mode of operation as the tool 10 of FIGS. 1-4 is illustrated in FIG. 7. The tool 10c has a shaft 14c with an expanded distal end portion 28c. The expanded portion 28c of the shaft 14c has an upwardly (as viewed in FIG. 7) facing major side surface 26c.

A gripper 32c is formed by a resilient wire 36c. A distal end portion of the wire 36c has a bend 100. The bend 100 may have any desired extent. In the embodiment of the present disclosure illustrated in FIG. 7, the bend 100 has an extent of almost 360° so that a loop 102 is formed in the distal end portion of the wire. The resiliently deflectable loop 102 is relatively large when it is in the unrestrained condition as illustrated in FIG. 7. The resilient loop 102 is compressed by pulling the wire 36c in a proximal direction, that is, toward the right as viewed in FIG. 7.

Rather than being a closed loop, as illustrated in FIG. 7, the loop 102 could have a U-shaped configuration and be open at the distal end of the loop. Alternatively, a series of loops may be formed at the distal end portion of the wire 36c.

The handle assembly 12 is illustrated in FIG. 8 and includes a pair of side sections 112 and 114. The side sections 112 and 114 are interconnected by suitable fasteners 116. The side sections 112 and 114 have recesses 120 to facilitate manual gripping of the side sections when they are interconnected by the fasteners 116.

The rocker member or actuator 38 is pivotally mounted on a shaft or pin 124 which extends between the side sections 112 and 114 (see FIG. 9). The rocker member or actuator 38 is received in recesses 128 and 130 (FIG. 8) formed in the side sections 112 and 114. The recesses enable the actuator 38 to be pivoted through a limited arcuate distance, in the manner illustrated schematically in FIG. 9, when the side sections 112 and 114 are fixedly interconnected by the fasteners 116.

The proximal end portion 16 of the shaft 14 is fixedly connected to an anchor block 136. The anchor block 136 is received in recesses 140 and 142 formed in the side sections 112 and 114. Two of the fasteners 116 extend through the anchor block 136 (FIG. 9) to secure the anchor block against movement relative to the side sections 112 and 114 of the handle.

The wire 36 extends through the shaft 14 and through the anchor block 136 (FIG. 9). The distal end of the wire 36 is fixedly connected to the actuator 38.

From the foregoing description, it is apparent that the present disclosure relates to new and improved method and apparatus for manipulating material 22 during a medical procedure. Material 22 to be positioned during the medical procedure is placed in engagement with a major side surface 26 on a distal end portion 18 of a shaft 14 which extends from a handle 12. A gripper 32 is movable along a longitudinal central axis of the shaft 14 to grip the material 22 between the gripper and the major side surface 26 of the distal end portion 18 of the shaft 14. A shoulder 64 may be provided between a distal end portion 18 of the shaft 14 and a proximally extending portion of the shaft 14 to retard movement of the material 22 in a proximal direction. A distal end portion 18 of the shaft 14 is shaped so that it can be used as a spatula for fine positioning of the material 22.

The following is claimed:

1. A tool for manipulating material during a medical procedure, the tool comprising:
a handle;
a shaft extending from the handle, said shaft having a distal end portion with a major side surface which is engagable with the material during the medical procedure; and
a gripper movable along a longitudinal central axis of said shaft to grip the material between said gripper and said major side surface of said distal end portion of said shaft.

2. A tool as set forth in claim 1 further including a shoulder which is disposed between said distal end portion of said shaft and a proximally extending portion of said shaft, said shoulder being engagable with the material to retard movement of the material in a proximal direction relative to said shaft.

3. A tool as set forth in claim 1 wherein said handle includes an actuator which is manually actuatable to move the gripper along the longitudinal central axis of the shaft.

4. A tool as set forth in claim 1 wherein said shaft is manually bendable to any one of a plurality of configurations to facilitate positioning of the material during the medical procedure.

5. A tool as set forth in claim 1 wherein said shaft has a tubular body which extends in a proximal direction from said distal end portion of said shaft, said distal end portion of said shaft being integrally formed as one piece with said tubular body, said gripper includes a wire which is at least partially disposed in said tubular body and is axially movable along said tubular body.

6. A tool as set forth in claim 5 wherein said wire extends from said tubular body and along said distal end portion of said shaft to enable the material to be gripped between said wire and said distal end portion of said shaft.

7. A tool as set forth in claim 6 wherein said wire is movable from an extended position extending along said distal end portion of said shaft toward said tubular body to decreases the extent to which said wire extends along said distal end portion of said shaft and to release the material for movement relative to the shaft.

8. A tool as set forth in claim 1 wherein said gripper includes a wire which is movable along said shaft and which has a distal end portion which extends across and is spaced from a portion of said major side surface of said distal end portion of said shaft to enable the material to be gripped between said distal end portion of said wire and said major side surface of said distal end portion of said shaft.

9. A tool as set forth in claim 1 said major side surface of said distal end portion of said shaft is flat.

10. A tool as set forth in claim 1 wherein said major side surface of said distal end portion of said shaft has an arcuately curving configuration as viewed in a plane extending perpendicular to the longitudinal central axis of said shaft.

11. A tool as set forth in claim 1 wherein a first portion of said major side surface of said distal end portion of said shaft has a flat configuration and a second portion of said major side surface of said distal end portion of said shaft has an arcuately curving configuration.

12. A tool as set forth in claim 1 wherein said gripper has a circular cross sectional configuration as viewed in a plane extending perpendicular to a longitudinal central axis of said gripper.

13. A tool as set forth in claim 1 wherein said gripper has an arcuately curving cross sectional configuration as viewed in a plane extending along the longitudinal central axis of said shaft.

14. A tool as set forth in claim 1 wherein said gripper has a distal end portion which curves away from said major side surface of said distal end portion of said shaft.

15. A tool as set forth in claim 1 wherein said gripper includes a loop which extends across a portion of said major side surface of said distal end portion of said shaft.

16. A method of positioning material at a target anatomical location during a medical procedure, the method comprising the steps of:
providing a tool that includes a handle, a shaft extending from the handle, and a gripping mechanism operably coupled to the handle, the shaft having a distal end portion;
placing the material on a first major surface of the distal end portion of the shaft,
operating the gripping mechanism to apply a linear engaging force to the material so that the material is securely retained on the first major surface;
advancing the material to the target anatomical location; and
operating the gripping mechanism to remove the linear engaging force from the material thereby deploy the material at the target anatomical location.

17. The method of claim 16, wherein a portion of the shaft is shaped by a user, prior to the advancing step, so that the portion is dimensioned to traverse one or more anatomical features.

18. The method of claim 16, wherein the surgical procedure is a robotic-assisted surgery.

19. The method of claim 16, wherein the surgical procedure is a cranio-facial surgical procedure.

20. The method of claim 16, wherein the linear engaging force is removed from the graft without displacing the graft from the first major surface.

21. A method of positioning material during a medical procedure, said method comprising the steps of:
positioning the material in engagement with a major side surface on a distal end portion of a shaft;
moving a distal end portion of a wire in a first direction along a longitudinal central axis of the shaft into engagement with the material to grip the material between the distal end portion of the wire and the major side surface of the distal end portion of the shaft;
moving the material to a desired location relative to a patient's body while the material is gripped between the distal end portion of the wire and the major side surface of the distal end portion of the shaft; and
moving the distal end portion of the wire in a second direction along the longitudinal central axis of the shaft to release the material for movement relative to the distal end portion of the shaft.
